SEQUENCE LISTING (2 sequences)

SEQ ID NO: 1 - protein, 7 aa, Homo sapiens
FRHDSGY

SEQ ID NO: 2 - protein, 21 aa, Homo sapiens
KTNMKHMAGAAAAGAVVGGLG

(19) United States
(12) Patent Application Publication
(10) Pub. No.: US 2004/0242845 A1
(43) Pub. Date: Dec. 2, 2004

(54) Methods and compositions comprising supramolecular antigenic constructs and antibodies elicited against them

(76) Inventors: Yves Claude Nicolau, Newton, MA (US); Ruth Greferath, Kehl (DE)

Correspondence Address: JOHN S. PRATT, ESQ, KILPATRICK STOCKTON, LLP, 1100 PEACHTREE STREET, ATLANTA, GA, 30309, US

(21) Appl. No.: 10/783,975

(22) Filed: Feb. 20, 2004

Related U.S. Application Data
(60) Provisional application No. 60/449,573, filed on Feb. 21, 2003.

(51) Int. Cl. G01N 33/53; C07K 1/00; C07K 14/00; C07K 17/00

(52) U.S. Cl. 530/350

(57) ABSTRACT

The present invention comprises novel compositions and methods for eliciting high immune responses, of great specifity yielding conformationally sensitive antibodies. These antibodies recognize specific epitopes on a wide variety of antigens including but not limited to, amyloid protein, prion protein, P₁₇₀ glycoprotein. The novel compositions of the invention comprise supramolecuIlar antigenic constructs generally comprising a peptide sequence, covalently attached to pegylated lysine resulting in modified and enhanced peptide presentation.

### FIELD OF THE INVENTION

The present invention is related to methods and compositions for eliciting high immune responses. In particular, the present invention includes novel compositions and methods for yielding conformationally sensitive antibodies.

### BACKGROUND OF THE INVENTION

Humoral immunity is mediated by serum antibodies which are proteins secreted by the B cell compartment of the immune response. Antibodies are a heterogeneous mixture of serum globulins, all of which share the ability to bind individually to specific antigens. All serum globulins with antibody activity are referred to as immunoglobulins.

All immunoglobulin molecules have common structural features which enable to do two things: 1) recognize and bind specifically to a unique structural entity on an antigen, and 2) perform a common biologic function after combining with the antigen. Basically, each immunoglobulin molecule consists of two identical light chains and two identical heavy chains linked by disulfide bridges.

A continuing need in the field of immunology and infectious disease, concerns the availability of highly specific and highly effective antibodies.

What is needed are effective methods and compositions for generation of highly specific and highly effective antibodies. Preferably such antibodies would recognize specific epitopes on various antigens such as amyloid protein, prion protein or P₁₇₀ glycoprotein.

### SUMMARY OF THE INVENTION

The present invention includes novel methods and compositions for eliciting highly specific and highly effective antibodies. Unlike currently available products the present invention provides unique methods and compositions resulting in antibodies having the ability to recognize specific epitopes from a range of antigens.

The present invention satisfies the long felt need for compositions that enable the generation of antibodies that specifically recognize epitopes such as those of amyloid protein, prion protein or P₁₇₀ glycoprotein.

The present invention comprises unique antigen presentation that results in enhanced exposure and ultimately antibodies with a higher degree of conformational sensitivity. In one embodiment the invention includes compositions comprising supramolectilar antigenic constructs comprising a peptide sequence, covalently attached to pegylated lysine-one at each terminus.

Accordingly, it is an object of the present invention to provide methods and compositions for eliciting specific and effective immune responses.

It is another object of the present invention to provide methods and compositions for treating and preventing the occurrence or spread of infectious disease.

It is a further object of the present invention to provide methods and compositions for preventing, treating or reducing disease by eliciting an active cellular and humoral response in the host.

Another object of the present invention is to provide methods and compositions for reducing and preventing the occurrence of hyperproliferative disorders.

It is yet another object of the present invention to provide methods and compositions for vaccinating a human or animal against selected infectious organisms.

It is yet another object of the present invention to provide methods and compositions for passively immunizing a human or animal against selected infectious organisms.

Another object of the present invention is to provide supramolecular antigenic construct compositions that are antigenic and elicit an immune response against infectious organisms in humans or animals.

Another object of the present invention is to provide vaccine compositions comprising supramolecular antigenic constructs that are non-immunogenic in a human or animal to be immunized with the composition; and carriers wherein the antigenic peptide is uniquely presented on the surface of the carrier such that resulting antibodies are highly specific and have a greater degree of conformational sensitivity when administered into the human or animal.

Yet another object of the present invention is to provide methods and compositions comprising modified antigenic moieties increase an individual's response to infectious disease.

It is yet another object of the present invention to provide immunogenic compositions wherein the carrier for the antigenic peptide comprises modified liposomes.

It is another object of the present invention to provide immunogenic compositions wherein the carrier for the antigenic peptide comprises a colloidal metal.

Another object of the present invention is to provide immunogenic compositions wherein the carrier for the antigenic peptide comprises a baculovirus-derived vesicle.

It is still another object of the present invention to provide immunogenic compositions in combination with pharmaceutically acceptable adjuvants to stimulate the immune response.

Yet another object of the present invention is to provide immunogenic compositions that may be administered intramuscularly, intravenously, transdermally, orally, or subcutaneously.

These and other objects, features and advantages of the present invention will become apparent after a review of the following detailed description of the disclosed embodiment and the appended claims.

### BRIEF DESCRIPTION OF THE FIGURES

FIG. 1 provides a schematic showing chemically modified β-amyloid antigen.

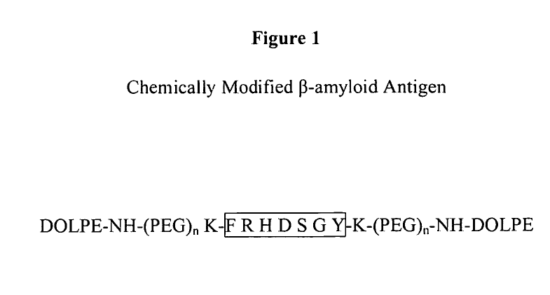

FIG. 2 provides representative schematic showing liposome reconstituted with a chemically modified amyloid-antigen.

FIG. 3 provides a schematic showing a multiple P₁₇₀ antigen.

FIG. 4 provides synthetic peptides, homologous to different segments of PrP^{c} used to investigate their influence on the viability of primary rat hippocampal neurons.

### DETAILED DESCRIPTION

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

We report here a method of eliciting high immune responses, of great specifity yielding conformationally sensitive antibodies. These antibodies recognize specific epitopes on a wide variety of antigens including but not limited to, amyloid protein, prion protein, P₁₇₀ glycoprotein.

Definitions

The terms “polypeptide”, “peptide”, and “protein”, as used herein, are interchangeable and are defined to mean a biomolecule composed of two or more amino acids linked by a peptide bond.

The term “peptides,” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (imino group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Typically, the amino acids making up a peptide are numbered in order, starting at the amino terminal and increasing in the direction toward the carboxy terminal of the peptide. Thus, when one amino acid is said to “follow” another, that amino acid is positioned closer to the carboxy terminal of the peptide than the preceding amino acid.

The term “residue” is used herein to refer to an amino acid that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

The phrase “consisting essentially of” is used herein to exclude any elements that would substantially alter the essential properties of the peptides to which the phrase refers. Thus, the description of a peptide “consisting essentially of . . . ” excludes any amino acid substitutions, additions, or deletions that would substantially alter the biological activity of that peptide.

Furthermore, one of skill will recognize that, as mentioned above, individual substitutions, deletions or additions which alter, add or delete a single amino acid or a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

The phrases “isolated” or “biologically pure” refer to material which is substantially or essentially free from components which normally accompany it as found in its native state. Thus, the peptides described herein do not contain materials normally associated with their in situ environment. Typically, the isolated, immunogenic peptides described herein are at least about 80% pure, usually at least about 90%, and preferably at least about 95% as measured by band intensity on a silver stained gel.

Protein purity or homogeneity may be indicated by a number of methods well known in the art, such as polyacrylamide gel electrophoresis of a protein sample, followed by visualization upon staining. For certain purposes high resolution will be needed and HPLC or a similar means for purification utilized.

When the immunogenic peptides are relatively short in length (i.e., less than about 50 amino acids), they are often synthesized using standard chemical peptide synthesis techniques.

Solid phase synthesis in which the C-terminal amino acid of the sequence is attached to an insoluble support followed by sequential addition of the remaining amino acids in the sequence is a preferred method for the chemical synthesis of the immunogenic peptides described herein. Techniques for solid phase synthesis are known to those skilled in the art.

Alternatively, the immunogenic peptides described herein are synthesized using recombinant nucleic acid methodology. Generally, this involves creating a nucleic acid sequence that encodes the peptide, placing the nucleic acid in an expression cassette under the control of a particular promoter, expressing the peptide in a host, isolating the expressed peptide or polypeptide and, if required, renaturing the peptide. Techniques sufficient to guide one of skill through such procedures are found in the literature.

Once expressed, recombinant peptides can be purified according to standard procedures, including ammonium sulfate precipitation, affinity columns, column chromatography, gel electrophoresis and the like. Substantially pure compositions of about 50 to 95% homogeneity are preferred, and 80 to 95% or greater homogeneity are most preferred for use as therapeutic agents.

One of skill in the art will recognize that after chemical synthesis, biological expression or purification, the immunogenic peptides may possess a conformation substantially different than the native conformations of the constituent peptides. In this case, it is often necessary to denature and reduce the antiproliferative peptide and then to cause the peptide to re-fold into the preferred conformation. Methods of reducing and denaturing proteins and inducing re-folding are well known to those of skill in the art.

Antigenicity of the purified protein may be confirmed, for example, by demonstrating reaction with immune serum, or with antisera produced against the protein itself.

The terms “a”, “an” and “the” as used herein are defined to mean “one or more” and include the plural unless the context is inappropriate.

The terms “detecting” or “detected” as used herein mean using known techniques for detection of biologic molecules such as immunochemical or histological methods and refer to qualitatively or quantitatively determining the presence or concentration of the biomolecule under investigation.

By “isolated” is meant a biological molecule free from at least some of the components with which it naturally occurs.

The terms “antibody” or “antibodies” as used herein include monoclonal antibodies, polyclonal, chimeric, single chain, bispecific, simianized, and humanized antibodies as well as Fab fragments, including the products of an Fab immunoglobulin expression library.

The term “antigen” refers to an entity or fragment thereof which can induce an immune response in a mammal. The term includes immunogens and regions responsible for antigenicity or antigenic determinants.

As used herein, the term “soluble” means partially or completely dissolved in an aqueous solution.

Also as used herein, the term “immunogenic” refers to substances which elicit or enhance the production of antibodies, T-cells and other reactive immune cells directed against an immunogenic agent and contribute to an immune response in humans or animals.

An immune response occurs when an individual produces sufficient antibodies, T-cells and other reactive immune cells against administered immunogenic compositions of the present invention to moderate or alleviate the disorder to be treated.

The term “carrier” as used herein means a structure in which antigenic peptide or supramolecular construct can be incorporated into or can be associated with, thereby presenting or exposing antigenic peptides or part of the peptide to the immune system of a human or animal. The term “carrier” further comprises methods of delivery wherein supramolecular antigenic construct compositions comprising the antigenic peptide may be transported to desired sites by delivery mechanisms. One example of such a delivery system utilizes colloidal metals such as colloidal gold.

In addition, the term “carrier” further comprises delivery mechanisms known to those skilled in the art including, but not limited to, keyhole limpet hemocyanin (KLH), bovine serum albumin (BSA) and other adjuvants. It is also to be understood that the supramolecular antigenic construct compositions of the present invention can further comprise adjuvants, preservatives, diluents, emulsifiers, stabilizers, and other components that are known and used in vaccines of the prior art. Any adjuvant system known in the art can be used in the composition of the present invention. Such adjuvants include, but are not limited to, Freund's incomplete adjuvant, Freund's complete adjuvant, polydispersed β-(1,4) linked acetylated mannan (“Acemannan”), TITERMAX® (polyoxyethylene-polyoxypropylene copolymer adjuvants from CytRx Corporation), modified lipid adjuvants from Chiron Corporation, saponin derivative adjuvants from Cambridge Biotech, killed Bordetella pertussis, the lipopolysaccharide (LPS) of gram-negative bacteria, large polymeric anions such as dextran sulfate, and inorganic gels such as alum, aluminum hydroxide, or aluminum phosphate.

Carrier proteins that can be used in the supramolecular antigenic construct compositions of the present invention include, but are not limited to, maltose binding protein “MBP”; bovine serum albumin “BSA”; keyhole lympet hemocyanin “KLH”; ovalbumin; flagellin; thyroglobulin; serum albumin of any species; gamma globulin of any species; syngeneic cells; syngeneic cells bearing la antigens; and polymers of D- and/or L-amino acids.

Further, the term “effective amount” refers to the amount of antigenic/immunogenic composition which, when administered to a human or animal, elicits an immune response. The effective amount is readily determined by one of skill in the art following routine procedures.

For example, supramolecular antigenic construct compositions may be administered parenterally or orally in a range of approximately 1.0 μg to 1.0 mg per patient, though this range is not intended to be limiting. The actual amount of the composition required to elicit an immune response will vary for each individual patient depending on the immunogenicity of the composition administered and on the immune response of the individual. Consequently, the specific amount administered to an individual will be determined by routine experimentation and based upon the training and experience of one skilled in the art.

The compositions of the present invention are used to produce antibodies directed against antigenic peptides. Resulting antibodies are administered to individuals to passively immunize them against disease or disorders such as Alzheimer's disease or prion disease.

The immunogenic compositions of the present invention comprise liposomes made by reconstituting liposomes in the presence of purified or partially purified or modified antigenic peptides. Additionally, peptide fragments may be reconstituted into liposomes. The present invention also includes antigenic peptide fragments modified so as to increase their antigenicity. For example, antigenic moieties and adjuvants may be attached to or admixed with the peptide. Examples of antigenic moieties and adjuvants include, but are not limited to, lipophilic muramyl dipeptide derivatives, nonionic block polymers, aluminum hydroxide or aluminum phosphate adjuvant, and mixtures thereof.

The present invention further encompasses antigenic peptides modified with hydrophobic moieties, such as palmitic acid, that facilitate insertion into the hydrophobic lipid bilayer of a carrier. Hydrophobic moieties of the present invention may be fatty acids, triglycerides and phospholipids wherein the fatty acid carbon back bones has at least 10 carbon atoms. Most preferable are lipophilic moieties having fatty acids with a carbon backbone of at least approximately 14 carbon atoms and up to approximately 24 carbon atoms. The most preferred hydrophobic moieties have a carbon backbone of at least 14 carbon atoms. Examples of hydrophobic moieties include, but are not limited to, palmitic acid, stearic acid, myristic acid, lauric acid, oleic acid, linoleic acid, and linolenic acid. The most preferred hydrophobic moiety is palmitic acid.

The supramolecular antigenic construct compositions of the present invention are administered to a human or animal to induce immunity to antigenic agents such as infectious organisms. The immunized human or animal develops circulating antibodies against the infectious organism, thereby reducing or inactivating its ability to stimulate disease.

The supramolecular antigenic construct compositions of the present invention are also used to produce a panel of monoclonal or polyclonal antibodies that are specific for various disorders. Antibodies are made by methods well known to those of ordinary skill in the art.

The compositions of the present invention are administered to a humliani or animal by any appropriate means, preferably by injection. For example, a modified antigenic peptide reconstituted in liposomes is administered by subcutaneous injection. Whether internally produced or provided from external sources, the circulating antibodies bind to antigen and reduce or inactivate its ability to stimulate disease.

Liposomes that can be used in the compositions of the present invention include those known to one skilled in the art. Any of the standard lipids useful for making liposomes may be used. Standard bilayer and multi-layer liposomes may be used to make compositions of the present invention. While any method of making liposomes known to one skilled in the art may be used, the most preferred liposomes are made according to the method of Alving et al., Infect. Imnmun. 60:2438-2444, 1992, hereby incorporated by reference. The liposome can optionally contain an adjuvant. A preferred adjuvant is detoxified lipid A, such as monophosphoryl or diphosphoryl lipid A.

When the vesicles are liposomes, the antigenic peptide generally has a hydrophobic tail that inserts into the liposome membrane as it is formed. Additionally, antigenic peptides can be modified to contain a hydrophobic tail so that it can be inserted into the liposome. For example, antigenic peptide may be exposed on the surface of previously formed liposomes by chemical attachment or electroinsertion.

The antibodies provided herein are monoclonal or polyclonal antibodies having binding specificity for infectious organisms or antigenic peptides representative of various disorders such as Alzheimer's disease, multi drug resistant cancer and prion diseases.

The monoclonal antibody is prepared by immunizing an animal, such as a mouse or rabbit, with supramolecular antigenic construct compositions of the present invention. Spleen cells are harvested from the immunized animals and hybridomas generated by fusing sensitized spleen cells with a myeloma cell line, such as murine SP2/O myeloma cells (ATCC, Manassas, Va.). The cells are induced to fuse by the addition of polyethylene glycol. Hybridomas are chemically selected by plating the cells in a selection medium containing hypoxanthine, aminopterin and thymidine (HAT).

Hybridomas are subsequently screened for the ability to produce monoclonal antibodies against specific diseases or disorders. Hybridomas producing antibodies of interest are cloned, expanded and stored frozen for future production. The preferred hybridoma produces a monoclonal antibody having the IgG isotype, more preferably the IgG1 isotype.

The polyclonal antibody is prepared by immunizing animals, such as mice or rabbits with supramolecular antigenic construct compositions of the present invention described above. Blood sera is subsequently collected from the animals, and antibodies in the sera screened for binding reactivity against target agents.

Either the monoclonal antibody or the polyclonal antibody, or both may be labeled directly with a detectable label for identification a target agent in a biological sample as described below. Labels for use in immunoassays are generally known to those skilled in the art and include enzymes, radioisotopes, and fluorescent, luminescent and chromogenic substances including colored particles, such as colloidal gold and latex beads. The antibodies may also be bound to a solid phase to facilitate separation of antibody-antigen complexes from non-reacted components in an immunoassay. Exemplary solid phase substances include, but are not limited to, microtiter plates, test tubes, magnetic, plastic or glass beads and slides. Methods for coupling antibodies to solid phases are well known to those skilled in the art.

Alternatively, the antibody may be labeled indirectly by reaction with labeled substances that have an affinity for imrmunoglobulin, such as protein A or G or second antibodies. The antibody may be conjugated with a second substance and detected with a labeled third substance having an affinity for the second substance conjugated to the antibody. For example, the antibody may be conjugated to biotin and the antibody-biotin conjugate detected using labeled avidin or streptavidin. Similarly, the antibody may be conjugated to a hapten and the antibody-hapten conjugate detected using labeled anti-hapten antibody. These and other methods of labeling antibodies and assay conjugates are well known to those skilled in the art.

In a preferred embodiment, the antibody is labeled indirectly by reactivity with a second antibody that has been labeled with a detectable label. The second antibody is preferably one that binds to antibodies of the animal from which the monoclonal antibody is derived. In other words, if the monoclonal antibody is a mouse antibody, then the labeled, second antibody is an anti-mouse antibody. For the monoclonal antibody to be used in the assay described below, this label is preferably an antibody-coated bead, particularly a magnetic bead. For the polyclonal antibody to be employed in the immunoassay described herein, the label is preferably a detectable molecule such as a radioactive, fluorescent or an electrochemiluminescent substance.

Formulations

The naturally occurring or synthetic protein, peptide, or protein fragment, containing all or an active portion of an immunogenic protein or peptide can be prepared in a physiologically acceptable formulation, such as in a pharmaceutically acceptable carrier, using known techniques. For example, the protein, peptide or protein fragment is combined with a pharmaceutically acceptable excipient to form a therapeutic composition.

Alternatively, the gene for the protein, peptide, or protein fragment, containing all or an active portion of the immunogenic peptide, may be delivered in a vector for continuous administration using gene therapy techniques. The vector may be administered in a vehicle having specificity for a target site, such as a tumor.

The compositions of the present invention may be administered in the form of a solid, liquid or aerosol. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for injection intramuscularly, subcutaneously, intravenously, intra-arterially, and formulations for topical and intraocular administration. Examples of aerosol formulations include inhaler formulations for administration to the lungs.

The compositions may be administered by standard routes of administration. In general, the composition may be administered by topical, oral, rectal, nasal or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the composition will depend on the condition being treated, the particular composition used, and other clinical factors such as weight and condition of the patient, and the route of administration.

The composition may be administered in combination with other compositions and procedures for the treatment of diseases. For example, unwanted cell proliferation may be treated conventionally with surgery, radiation or chemotherapy in combination with the administration of the composition, and additional doses of the composition may be subsequently administered to the patient to stabilize and inhibit the growth of any residual unwanted cell proliferation.

Supramolecular Antigenic Constructs The supramolecular antigenic constructs of the present invention generally comprise a peptide sequence, covalently attached to pegylated lysine-one at each terminus. The length of the PEG (polyethylenglycol) chain may vary from 8 to 150000. The free PEG terminus is covalently attached to a molecule of phosphatidylethanolamine (where the fatty acid can be: myristic, palmitic, stearic, oleic etc. or combination thereof). This supramolecular structure is reconstituted in liposomes consisting of phospholipids and cholesterol (phosphatidylethanol amine, phosphatidyl glycerol, cholesterol in varied molar ratios. Other phospholipids can be used. Lipid A is used at a concentration of approximately 40 μg/pmole of phospholipids.

In certain embodiments, the supramolecular antigenic constructs comprise a peptide having the amino acid sequence of f3-amyloid. In certain other embodiments, the supramolecular 1 antigenic construct comprises peptide sequences that are the extracellular loops 1, 4 and 6 of the P170 glycoprotein. In certain other embodiments, the supramolecular 1 antigenic construct comprises peptide sequences that comprise amino acid sequences 109-129 of the prion protein.

The present invention further comprises monoclonal antibodies raised against a supramolecular structure reconstituted in liposome, wherein, for example, the peptide sequence comprises an amino acid sequence from t3-amyloid protein. Additionally, monoclonal antibodies raised against supramolecular structures wherein the peptide sequence is an/or several amino acid sequences from the P-glycoprotein (P₁₇₀) extracellular loops are also included in the present invention.

Also included in the present invention are monoclonal antibodies raised against a supramolecular structure wherein the peptide sequences comprise an amino acid sequence selected from a protein of interest. More specifically, for example, the invention includes monoclonal antibodies raised against a supramolecular structure reconstituted in liposome wherein the peptide sequence is an amino acid sequence selected from 13-amyloid protein (4 -10, or 1 -8, or 8 -16, etc.) which does not induce cerebral bleeding in transgenic mice for human Alzheimer's disease. The invention further includes monoclonal antibodies sensitive to the conformational characteristics of antigenic peptides.

Amyloid

The 7 amino acid sequence: FRHDSGY (SEQ ID NO:1) of β-amyloid was synthesized. One lysine was attached covalently at each end of the sequence (1). The lysines, prior to attachment to the above sequence were reacted with a chain of Polyethylenglycol (PEG, n=8-2000). Polyethylenglycol chains bound to lysine at one end are covalently attached to a molecule of dioleyl-phosphatidyl choline ethanolamine (or any fatty acid—phosphatidylcholine) as described (2).

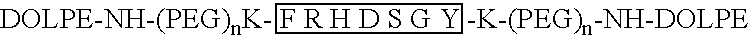

### Chemically Modified P-Amyloid Antigen

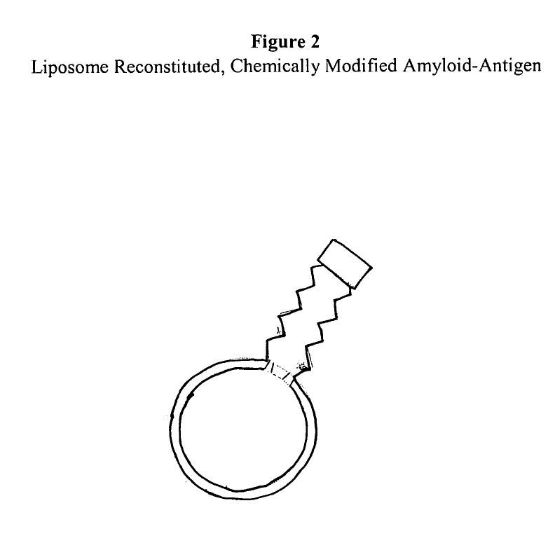

The chemically modified antigen is then reconstituted in liposomes consisting of phospholipids and cholesterol (3). Examples of suitable liposomes include, but are not limited to, DOPG, DOPEA, Chol. (Lipid A was at the concentration of 40 μg/μmole phospholipid.) A representative schematic showing liposome reconstituted with a chemically modified amyloid-antigen is shown in FIG. 2.

The supramolecular antigenic constructs of the present invention have vast advantages over the palmitoylated antigens, reconstituted in liposomes:

1. The long PEG chains (n =8 -5000) enhance significantly the exposure and accessibility of the peptide sequence. Antigen presentation becomes much more and the conformation sensitivity of the elicited antibodies is enhanced.

2. Peptides sequences in different conformations may be used. The increased distance between the sequence and surface of the liposome makes sure that the surface does not interact with the sequence, thus, possibly influencing its conformation.

3. Antigenicity of the construct becomes significantly higher than that of palmitoylated sequences reconstituted in liposomes. High titers of antibodies comprised between 1:5000 and 1:0000 are obtained in mice, within a few weeks.

4. Affinity of the antibodies for the antigen is significantly increased. In the case of the amyloid sequence FRHDSGY, the antibody elicited by ip or iv injection of the construct are efficiently solubilizing Aβ₁₋₄₀ and Aβ₁₋₄₂fibers, protecting in vitro PC12 cells against apoptosis and metabolic inhibition (MTT reduction) induced by Aβ₁₋₄₂ and Aβ₁₋₄₀ fibers.

Any other sequence of the amyloid protein can be substituted to the FRHDSGY sequence. Monoclonal antibodies obtained from mice immunized with the described construct display, besides the in vitro properties mentioned above for the polyclonal antibodies-biological activity in APP[V7171] FVB transgenic mice for human Alzheimer's Disease. Significant levels of memory restoration and of curiosity awakening in these mice are observed. The mAb does not induce bleeding in the brain of the immunized, transgenic mice. Though not wishing to be bound by the following theory, the antibodies appear to bind preferentially to the a-helix conformation of the amyloid protein.

Multidrug Resistance 1 (MDR 1) in Cancer Cells

Multidrug resistance 1 in cancer cells is caused by the overexpression of the P-glycoprotein (P₁₇₀), a membrane pump which ejects a large variety of unrelated chemotherapy agents from cancer cells.

Immunization with palmitoylated extracellular sequences of P₁₇₀, reconstituted in liposomes, led to restoration of the sensitive phenotype in vitro in MDR1 L₁₂₁₀ mouse leukemia cells (3). Further results have been obtained in vivo (Madoulet, Tosi, Nicolau et al., 2002 -unpublished results) indicating a 70% increase of survival half-life in immunized mice, inoculated with MDR cancer cells, undergoing chemotherapy.

The inventors of the present invention demonstrate herein that an antigen consisting of the P₁₇₀ extracellular sequences 1,4 and 6 constructed, according to the method of the present invention is far more efficient in eliciting antibodies which largely revert the MDR phenotype to the sensitive phenotype in vitro and in vivo.

According to the methods of the present invention, peptides corresponding to P₁₇₀ extracellular loops 1, 4 and 6 were synthesized and then attached to pegylated lysines-1 at each end-which in turn were covalently attached to one dioleyl phosphatylethanolamine molecule at each end. Any fatty acid, myristic, palmitic, stearic or polyunsaturated fatty acids may be used.

These 3 constructs were reconstituted in liposomes consisting of PC-PEA-PG-Cholesterol (or any other phospholipid and cholesterol combination). Lipid A was added at concentration of 40 μg/lmole of phospholipids. The ratio peptide: phospholipid was 1:200 (other ratios may be used).

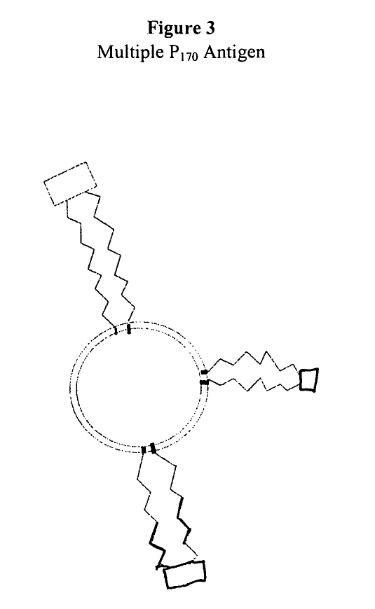

The length of the polyethylenglycol chains varied: the longer the peptide sequence, the higher the number of PEG molecules in the chain needs to be. For the 3 sequences used, the PEG chain-length varied from 10 to 5000. Other chain lengths can be used. FIG. 3 provides a representative schematic showing a multiple P₁₇₀ antigen.

IP inoculation of this antigen, followed by three boostings at 2 weeks interval elicited high titres of anti P₁₇₀ antibodies (1:5000-1: 10000) capable of blocking the pumping activity of P₁₇₀, in vitro and in vivo.

Prion Diseases

Prions cause neurodegenerative diseases such as scrapie in sheep, bovine spongiform encephalopathy in cattle and Creutzfeldt-Jacob-Disease in humans. The only known component of the particle is the scrapie isoform of the protein, PrP^{Sc}. Although prions multiply, there is no evidence that they contain nucleic acid. PrP^{Sc} is derived from the non-infectious, cellular protein PrP^{c} by a posttranslational process during which PrP^{c} undergoes a profound conformational change.

The scrapie protein, PrP^{Sc} has a critical role in neuronal degeneration and during disease development undergoes a three stage transition as follows: (normal cellular isoform of protein) PrP^{c}—infectious form (scrapie isoform of protein) PrP^{Sc}_protein PrP27-30. Such a cascade of events occurs during the development of Creutzfeldt—Jacob Disease (CJD), Kuru, Gerstmann—Straussler-Scheinker Syndrome (GSS), fatal familial insomnia in man, scrapie in sheep and goats, encephalopathy in mink and bovine spongiform encephalopathy in cattle.

The cellular non-toxic protein (PrP^{c} is a sialoglycoprotein of MW 33-35 K that is expressed predominantly in neurons. In the diseases mentioned above, PrP^{c} is converted into an altered form (PrP^{Sc}), which is distinguishable from its normal homologue by its relative resistance to protease digestion. PrP^{Sc} accumulates in the central nervous system of affected animals and individuals and its protease-resistant core aggregates extracellularly. The molecular basis of the pathogenesis is not understood.

Very interesting observations were made concerning the neurotoxicity of a fragment of the protein, which may have a bearing on the understanding of the mechanism of nerve cell-degeneration occurring in related encephalopaties.

On the basis of the observation, that the (J-amyloid fragment responsible for the extracellular deposition of amyloid fibrils and plaques in the Alzheimer Disease is neurotoxic, it was hypothesized that neuronal death in related encephalopathies might be due to toxic effects of abnormal extracellular accumulation of PrP^{Sc} and/or its degradation products.

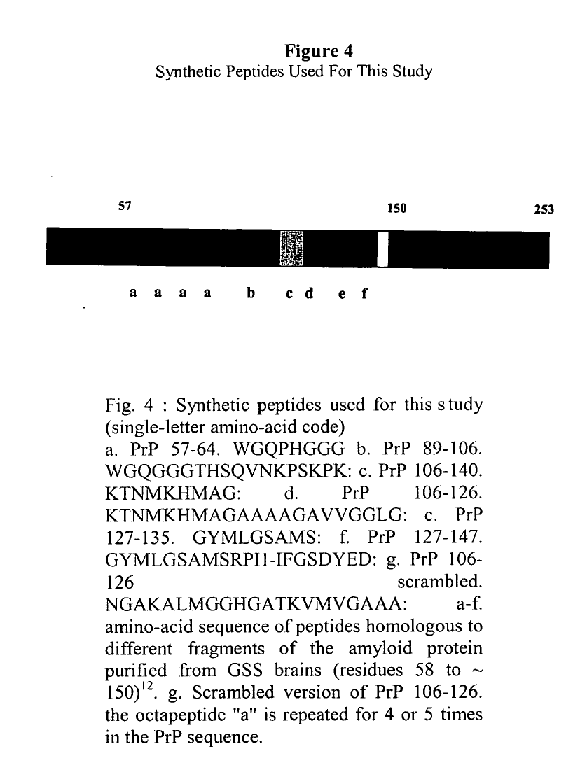

Synthetic peptides, homologous to different segments of PrP^{c} were used to investigate their influence on the viability of primary rat hippocampal neurons (FIG. 4)

The present inventors demonstrated that neuronal death occurs from chronic exposure of primary rat hippocampal cultures to micromolar concentrations of a peptide corresponding to residues 106126 of the amino-acid sequence deduced from human PrP^{c} cDNA, in a concentration dependent manner (Example 1).

As detailed in Example 1, the inventors showed that the neuronal death induced by PrP 106-126 occurred by apoptosis in a dose dependent manner. In the terminal stages of subacute encephalopaties, such as scrapie, PrP^{Sc} reaches at whole brain concentrations 10 to 20 times higher than PrP^{c}, which resembles strikingly the data listed in Table 1 for the 2 concentrations of PrP106-126.

The process of programmed cell death induced by PrP106-126 is associated, among others with the induction of the testosterone-repressed prostate message-2 gene (TRPM-2). It is not known whether apoptosis is activated in vivo during-related encephalopaties, but the expression of the TRPM-2 mRNA is increased 10-fold in scrapie-infected hamsters.

It appears from these data, that a neurotoxic mechanism is possibly responsible for neuronal cell loss in related encephalopaties and could also be relevant in Alzheimer's disease.

The possible mechanism of this neurotoxicity was investigated in a model system aiming at detecting and analyzing ionic channel formations upon the interaction of peptides or proteins with lipid bilayers.

Low pH, which favors channel formation by PrP106-126, converts also this peptide from ahelical to R-sheet conformation. Whereas peptide mapping of PrP^{Sc} with Edman sequencing and mass spectrometry revealed no differences between its amino acid sequence and that predicted from the PrP^{c} gene sequence; no chemical modifications where found that might distinguish PrP^{Sc} from PrP^{P}; Fourier Transform infrared spectroscopy and circular dichroism spectroscopy revealed however a significant conformational difference between PrP^{Sc} and PrP^{P}.

PrP^{c} is essentially a-helical with little or no R-sheet, whereas PrP^{Sc} has a high β-sheet content and less α-helical structure.

The sequence KTNMKHMAGAAAAGAVVGGLG (PrP106-126) is not only very hydrophobic but it converts also, at low pH to β-sheet conformnation. Moreover, it can convert in solution, other peptides to β-sheet conformation.

Based upon these observations and upon techniques developed by the inventors, a “vaccine” was developed against diseases by eliciting a strong humoral and cellular immune response in mice to the neurotoxic PrP106-126, and then challenge the immunized mice with brain extracts from scrapie mice.

As in the previous examples, pegylated lysines were attached covalently at each end of the PrP106-126 sequence. The length of the PEG chain was 12 -4000. The PEG chains were coupled each other to one molecule of phosphatidyl ethanol amine and reconstituted in PG-PEA-chol liposomes-lipid A.

Injected into mice these supramolecular antigenic constracts elicited a strong humoral immune response, yielding antibodies with high affinity for the PrP106-126 sequence, and having solubilizing effects within.

It should be understood that the foregoing relates only to preferred embodiments of the present invention, and that numerous modifications or alterations may be made therein without departing from the spirit and the scope of the invention as set forth in the appended claims. The references cited throughout are hereby incorporated by reference in their entireties.

### EXAMPLE 1

The present inventors demonstrated that neuronal death occurs from chronic exposure of primary rat hippocampal cultures to micromolar concentrations of a peptide corresponding to residues 106126 of the amino-acid sequence deduced from human PrP^{c} cDNA, in a concentration dependent manner. The data are shown in Table 1.

TABLE 1
Chronic treatment of hippocampal neurons for 9 days

| | cell death % | |
|---|---|---|
| Peptide | 20 μm | 80 μm |
| PrP 106-126 | 18 ± 8 | 100 ± 8 |
| PrP 57-64 | 0 ± 5 | 3 ± 4 |
| PrP 89-106 | 5 ± 2 | 2 ± 6 |
| PrP 106-114 | 0 ± 3 | 12 ± 6 |
| PrP 127-135 | 3 ± 6 | 15 ± 9 |
| PrP 127-147 | 1 ± 7 | 18 ± 7 |
| PrP 106-126 scrambled | 3 ± 2 | 8 ± 3 |

The data are the means ± s.e. of 6-10 determinations and are normalized to the toxic effect of PrP106-126 (designated 100% response).

It was shown that the neuronal death induced by PrP 106-126 occurred by apoptosis in a dose dependent manner. In the terminal stages of subacute encephalopaties, such as scrapie, PrP^{Sc} reaches at whole brain concentrations 10 to 20 times higher than PrP^{c}, which resembles strikingly the data listed in Table 1 for the 2 concentrations of PrP106-126.

The process of programmed cell death induced by PrP106-126 is associated, among others with the induction of the testosterone-repressed prostate message-2 gene (TRPM-2). It is not known whether apoptosis is activated in vivo during-related encephalopaties, but the expression of the TRPM-2 mRNA is increased 10-fold in scrapie-infected hamsters.

### References

1. C. Nicolau, R. Greferath, T. S. Balaban, J. Lazarte and R.

2. Fluka A G (2002) Cat. # 79898.

3. P. -F. Tosi, D. Radu, and C. Nicolau (1995). Biochem. Biophys. Res. Chem. 212, 494-500.

We claim:

1. A composition comprising supramolecular antigenic constructs.

2. The composition of claim 1, wherein the supramolecular antigenic constructs comprises:
a peptide sequence, covalently attached to pegylated lysine-one at each terminus;
wherein the free PEG terminus is covalently attached to a molecule of phosphatidylethanolamine.

3. The composition of claim 2, wherein the supramolecular structure is reconstituted in liposomes consisting of phospholipids and cholesterol.

4. The composition of claim 1, wherein the supramolecular antigenic constructs may be used to treat disorders comprising Alzheimer's disease, multidrug resistance in cancer cells, or prion diseases.
